(19) United States
(12) Patent Application Publication
(10) Pub. No.: US 2015/0005594 A1
(43) Pub. Date: Jan. 1, 2015

(54) SYSTEM AND METHOD TO DETERMINE Sp02 VARIABILITY AND ADDITIONAL PHYSIOLOGICAL PARAMETERS TO DETECT PATIENT STATUS

(71) Applicant: Covidien LP, Mansfield, MA (US)

(72) Inventors: Nassib G. Chamoun, Needham, MA (US); Jeffrey C. Sigl, Medway, MA (US); Scott D. Greenwald, Medfield, MA (US); Paul J. Manberg, Newton, MA (US)

(21) Appl. No.: 14/490,498

(22) Filed: Sep. 18, 2014

Related U.S. Application Data
(63) Continuation of application No. 13/212,874, filed on Aug. 18, 2011.
(60) Provisional application No. 61/376,482, filed on Aug. 24, 2010.

(51) Int. Cl. A61B 5/1455; A61B 5/00; A61B 5/021

(52) U.S. Cl. CPC A61B 5/14552; A61B 5/021; A61B 5/4821
USPC 600/301; 600/324

(57) ABSTRACT

Systems and methods for detecting untoward clinical states (e.g., hypoperfusion) and classifying patient state based on at least one calculated physiological parameter are provided. The patient state classification may be used by a physician to determine patient condition and relative risk to guide decision making during a procedure.

### CROSS REFERENCE TO RELATED APPLICATION

This application is a continuation of U.S. patent application Ser. No. 13/212,874 filed Aug. 18, 2011, which is hereby incorporated by reference herein in its entirety, and which claims benefit of Provisional Application Ser. No. 61/376,482, filed on Aug. 24, 2010.

### SUMMARY OF THE DISCLOSURE

Systems and methods for improving patient outcomes and, more particularly, for using SpO2 variability and other physiological parameters to detect patient status are disclosed. In some embodiments, these systems and methods may use SpO2 variability and other physiological parameters to detect hypoperfusion. Such systems and methods may allow for a clinician to provide earlier interventions that improve patient outcomes.

Patient management may use real-time or near real-time clinical data and physiological measures to estimate the patient's condition and/or clinical state. For example, hypoperfusion in a patient may be identified based on various physiological parameters and treated accordingly. In this example, clinical management decisions can be made based on the patient's adequacy of perfusion. Medical interventions, such as vasopressor administration, fluid administration, increasing heart rate and heart contractility or anesthetic titration which occur soon after a patient enters a clinical state associated with hypoperfusion, may yield better outcomes than medical interventions made after a patient has spent a longer time in this clinical state. Consequently, a clinical decision support system is desired in order to provide alerts within a clinically desired time after patients enter an undesired state such as a state of hypoperfusion. This support system may be designed to help physicians achieve improved patient outcomes.

According to one aspect, the disclosure relates to a method for monitoring a patient. For example, the method may include collecting physiological data from a patient using at least one sensor, and delivering the physiological data to a processor to calculate at least one physiological parameter based at least in part on the physiological data, and classify the patient as being in a hypoperfusion state based at least in part on the at least one physiological parameter. In certain embodiments, collecting the physiological data occurs non-invasively. In certain embodiments, calculating at least one physiological parameter includes performing a statistical operation on the physiological data. The at least one physiological parameter can include, for example, at least one of SpO2, SpO2 variability, SpO2 Range, EEG, BIS and Mean Arterial Pressure. In some embodiments, classifying the patient as being in a hypoperfusion state includes comparing the at least one physiological parameter to a parameter threshold. In certain embodiments, the parameter threshold is determined from population data. According to one aspect, the method includes combining two or more physiological parameters to classify the patient as being in a hypoperfusion state.

In an embodiment, the method includes displaying at least one of an indication of the hypoperfusion state and a physiological parameter on a display. Some embodiments include displaying said at least one physiological parameter and the indication of hypoperfusion state in real-time. In one aspect, an alarm is activated to indicate the patient state classification. In certain embodiments, a patient risk of reaching an endpoint based at least in part on the at least one physiological parameter is determined.

In an embodiment a system for monitoring a patient includes at least one sensor capable of collecting physiological data from a patient, a processor configured to use at least a portion of the physiological data to: calculate a parameter indicative of the patient's oxygen saturation variability based at least in part on the physiological data, determine a patient state based at least in part on the oxygen saturation variability parameter, and calculate a risk assessment of the patient based at least in part on the determined patient state. In certain embodiments, the system includes a display operative to show said indication of the risk assessment.

In an embodiment, the processor is configured to receive at least one of the patient's medical history, demographic information and a population database. In certain embodiments, the risk assessment is calculated from a combination of the patient's medical history and a calculated physiological parameter. In some embodiments the risk assessment is calculated from a combination of a population databases and a calculated physiological parameter.

In one aspect, the processor is further capable of calculating a reference set from the received input, defining a plurality of patient states from the reference set, providing an endpoint, calculating risk parameters associated with the endpoint for each of the plurality of patient states, and calculating a patient state based at least in part on the combination of data, wherein the risk assessment is based at least in part on the calculated patient state and the calculated risks. In certain embodiments, risks are calculated based at least in part on a Cox Regression model.

In one aspect, the processor is capable of using at least a portion of the physiological data to: calculate a parameter indicative of the patient's brain state based at least in part on the physiological data, and determine the patient state based at least in part on the brain state parameter, the oxygen saturation variability parameter, and a population database. In certain embodiments, the processor is capable of using at least a portion of the physiological data to calculate a parameter indicative of the patient's blood pressure, and determine the patient state based at least in part on the blood pressure parameter, the oxygen saturation variability parameter, and a population database.

In an embodiment a method for monitoring a patient includes collecting at least two of SpO2, BIS and MAP data from a patient, determining a patient state based at least in part on the collected data, and displaying at least one of the patient state and the collected data on a display.

### BRIEF DESCRIPTION OF THE DRAWINGS

The above and other features will be more apparent upon consideration of the following detailed description, taken in conjunction with the accompanying drawings in which:

FIG. 1 shows an illustrative pulse oximetry system in accordance with an embodiment;

FIG. 2 is a block diagram of the illustrative pulse oximetry system of FIG. 1 coupled to a patient in accordance with an embodiment;

FIG. 3 shows a schematic view of system for detecting brain state in accordance with an embodiment;

FIG. 4 shows an illustrative block diagram of a patient monitoring system capable of monitoring a patient according to an embodiment;

FIG. 5 is a flow chart of illustrative steps involved in classifying and displaying a patient state according to an embodiment;

FIG. 6 shows illustrative displays of patient state and risk assessment information according to an embodiment;

FIGS. 7A-7E show illustrative plots of data related to oxygen saturation and other physiological parameters according to embodiments; and

FIG. 8 shows an illustrative chart of the relative risk hazards for each patient state based on various physiological parameters according to an embodiment.

### DETAILED DESCRIPTION

A processor in a monitoring system collects physiological data from a patient over a set time period. Patient physiological data includes, for example, information that can be measured from a patient (e.g., heart rate (HR), respiratory rate, blood pressure (BP—Mean Arterial Pressure (MAP), Systolic Pressure, Diastolic Pressure), as well as derived hemodynamic parameters (ratios, products or differences of heart rate and the components of BP, e.g., Systolic/Diastolic or MAP/HR), Bispectral Index™ (BIS™), SpO2, temperature, ScO2, rSO2, etc.) and information about patient interventions (e.g., the start of a surgical procedure, intubation of the patient, the administration of drugs, etc.). This data may be collected in real-time, or at any other clinically appropriate interval. A technique for improving patient outcomes based on a “Triple Low” of BIS, MAP and MAC is described in U.S. patent application Ser. No. 12/752,288 filed Apr. 1, 2010 and entitled “System and Method for Integrating Clinical Information to Provide Real-Time Alerts for Improving Patient Outcomes,” and U.S. Provisional Application No. 61/165,672 filed Apr. 1, 2009 and entitled “System and Method for Integrating Clinical Information to Provide Real-Time Alerts for Improving Patient Outcomes,” which are hereby incorporated by reference in their entirety. The processor may calculate one or more physiological parameters based at least in part on the collected physiological data. The physiological data and physiological parameters may be used at least in part by the monitoring system to classify the patient as being in one of a plurality of states that may guide the decision making of a physician. At least one of the physiological data, the physiological parameters and the classification of the patient state may be provided to and displayed by the monitoring system that may allow a risk-assessment to be provided to the physician.

The provision of a patient classification may allow a physician to make decisions sooner and better with more information. The monitoring system may provide alarms to alert the physician to a patient entering an undesirable state at any given moment. The monitoring system may further alert the physician that this undesirable state is associated with a particular outcome. The monitoring system may also indicate to the physician one or more changes that can be made that may cause the patient to enter a more desirable state.

The monitoring system may display a scale or other indication related to a patient's adequacy of cerebral or systemic perfusion, as reflected, for example, by their SpO2 level, Bispectral Index™ level, blood pressure and heart rate. Systemic perfusion relates to the amount of nutrient delivery of arterial blood in a patient's organs. Cerebral perfusion relates to the amount of nutrient delivery of arterial blood in a patient's brain. Systemic or cerebral hypoperfusion may occur as a result of low blood pressure or low circulating blood volume. Consequences of hypoperfusion include inadequate oxygen delivery, poor removal of cellular waste, or both conditions. Inappropriately high levels of anesthetic or other agents may result in blood pressures and/or heart rates too low to ensure an adequate supply of oxygen to the brain and other end organs. This inadequate supply of oxygen to the brain and other end organs may be reflected in Bispectral Index values that are lower than expected for a given anesthetic agent dose and in lower SpO2 levels. For ease of illustration, systemic and/or cerebral hypoperfusion will both be referred to herein as hypoperfusion. An alarm may indicate that the patient is in a state of hypoperfusion and that this state is associated with increased mortality. Similarly, the system may display a plurality of other information such as the variability of a patient's oxygen saturation, or a patient's average blood pressure over a period of time. The physician may then provide an intervention for the patient based at least in part on the displayed information to help place the patient in a more desirable state.

Systemic and cerebral perfusion levels may vary from local tissue perfusion, e.g., of a finger or body part. While local perfusion may be measured (e.g., using a pulse oximetry device), there has previously been no effective way to detect systemic and cerebral hypoperfusion non-invasively. For example, previous systems have measured local perfusion at one or more tissue sites, however this technique has been inadequate at evaluating systemic or cerebral perfusion.

The monitoring system may collect the physiological data using at least one sensor (or sensor system) capable of collecting physiological data from a patient. For example, the one or more sensors may include a pulse oximetry system for measuring the oxygen saturation of a patient's blood. The one or more sensors may include an EEG acquisition apparatus for measuring a patient's brain state, for example by calculating the Bispectral Index™ (BIS™). Other sensors which may be used in the monitoring system include those associated with cerebral or somatic oximetry monitors, blood pressure monitors, heart rate monitors, and monitors of hemodynamic parameters such as stroke volume (SV), pulse pressure (PP), cardiac output (CO), stroke volume variability (SVV), and pulse pressure variability (PPV).

An oximeter is a medical device that may determine the oxygen saturation of the blood. One common type of oximeter is a pulse oximeter, which may indirectly measure the oxygen saturation of a patient's blood (as opposed to measuring oxygen saturation directly by analyzing a blood sample taken from the patient) and changes in blood volume in the skin. Ancillary to the blood oxygen saturation measurement, pulse oximeters may also be used to measure the pulse rate of the patient. Pulse oximeters typically measure and display various blood flow characteristics including, but not limited to, the oxygen saturation of hemoglobin in arterial blood.

An oximeter may include a light sensor that is placed at a site on a patient, typically a fingertip, toe, forehead or earlobe, or in the case of a neonate, across a foot. The oximeter may pass light using a light source through blood perfused tissue and photoelectrically sense the absorption of light in the tissue. For example, the oximeter may measure the intensity of light that is received at the light sensor as a function of time. A signal representing light intensity versus time or a mathematical manipulation of this signal (e.g., a scaled version thereof, a log taken thereof, a scaled version of a log taken thereof, etc.) may be referred to as the photoplethysmograph (PPG) signal. In addition, the term “PPG signal,” as used herein, may also refer to an absorption signal (i.e., representing the amount of light absorbed by the tissue) or any suitable mathematical manipulation thereof. The light intensity or the amount of light absorbed may then be used to calculate the amount of the blood constituent (e.g., oxyhemoglobin) being measured as well as the pulse rate and when each individual pulse occurs.

The light passed through the tissue is selected to be of one or more wavelengths that are absorbed by the blood in an amount representative of the amount of the blood constituent of interest present in the blood. The amount of light passed through the tissue varies in accordance with the changing amount of blood constituent in the tissue and the related light absorption. Red and infrared wavelengths may be used because it has been observed that highly oxygenated blood will absorb relatively less red light and more infrared light than blood with a lower oxygen saturation. By comparing the intensities of two wavelengths at different points in the pulse cycle, it is possible to estimate the blood oxygen saturation of hemoglobin in arterial blood.

When the measured blood parameter is the oxygen saturation of hemoglobin, a convenient starting point assumes a saturation calculation based on Lambert-Beer's law. The following notation will be used herein:

I(λ,t)=Iₒ(λ)exp(−(sβₒ(λ)+(1−s)βᵣ(λ)l(t) (1)

where:
λ=wavelength;
t=time;
I=intensity of light detected;
Iₒ=intensity of light transmitted;
s=oxygen saturation;
βₒ, βᵣ=empirically derived absorption coefficients; and
l(t)=a combination of concentration and path length from emitter to detector as a function of time.

One approach measures light absorption at two wavelengths (e.g., red and infrared (IR)), and then calculates saturation by solving for the “ratio of ratios” as follows.

1. First, the natural logarithm of (1) is taken (“log” will be used to represent the natural logarithm) for IR and Red

log I=log Iₒ−(sβₒ+(1−s)βᵣ)l (2)

2. (2) is then differentiated with respect to time

$$\frac{d log I}{d t} = - ( s {β}_{o} + ( 1 - s ) {β}_{r} ) \frac{d l}{d t} \qquad ( 3 )$$

3. Red (3) is divided by IR (3)

$$\frac{d log I ( {λ}_{R} ) \text{/} d t}{d log I ( {λ}_{IR} ) \text{/} d t} = \frac{s {β}_{o} ( {λ}_{R} ) + ( 1 - s ) {β}_{r} ( {λ}_{R} )}{s {β}_{o} ( {λ}_{IR} ) + ( 1 - s ) {β}_{r} ( {λ}_{IR} )} \qquad ( 4 )$$

### 4. Solving for s

$$s = \frac{\frac{d log I ( {λ}_{IR} )}{d t} {β}_{r} ( {λ}_{R} ) - \frac{d log I ( {λ}_{R} )}{d t} {β}_{r} ( {λ}_{IR} )}{\frac{d log I ( {λ}_{R} )}{d t} ( {β}_{o} ( {λ}_{IR} ) - {β}_{r} ( {λ}_{IR} ) ) - \frac{d log I ( {λ}_{IR} )}{d t} ( {β}_{o} ( {λ}_{R} ) - {β}_{r} ( {λ}_{R} ) )}$$

Note in discrete time

$$\frac{d log I ( λ , t )}{d t} ≃ log I ( λ , {t}_{2} ) - log I ( λ , {t}_{1} )$$

### Using log A−log B=log A/B,

$$\frac{d log I ( λ , t )}{d t} ≃ log ( \frac{I ( {t}_{2} , λ )}{I ( {t}_{1} , λ )} )$$

So, (4) can be rewritten as

$$\frac{\frac{d log I ( {λ}_{R} )}{d t}}{\frac{d log I ( {λ}_{IR} )}{d t}} ≃ \frac{log ( \frac{I ( {t}_{1} , {λ}_{R} )}{I ( {t}_{2} , {λ}_{R} )} )}{log ( \frac{I ( {t}_{1} , {λ}_{IR} )}{I ( {t}_{2} , {λ}_{IR} )} )} = R \qquad ( 5 )$$

where R represents the “ratio of ratios.” Solving (4) for s using (5) gives

$$s = \frac{{β}_{r} ( {λ}_{R} ) - R {β}_{r} ( {λ}_{IR} )}{R ( {β}_{o} ( {λ}_{IR} ) - {β}_{r} ( {λ}_{IR} ) ) - {β}_{o} ( {λ}_{R} ) + {β}_{r} ( {λ}_{R} )} .$$

From (5), R can be calculated using two points (e.g., PPG maximum and minimum), or a family of points. One method using a family of points uses a modified version of (5). Using the relationship

$$\frac{d log I}{d t} = \frac{d I \text{/} d t}{d I} \qquad ( 6 )$$

now (5) becomes

$$\begin{matrix}\frac{\frac{d log I ( {λ}_{R} )}{d t}}{\frac{d log I ( {λ}_{IR} )}{d t}} ≃ \frac{\frac{I ( {t}_{2} , {λ}_{R} ) - I ( {t}_{1} , {λ}_{R} )}{I ( {t}_{1} , {λ}_{R} )}}{\frac{I ( {t}_{2} , {λ}_{IR} ) - I ( {t}_{1} , {λ}_{IR} )}{I ( {t}_{1} , {λ}_{IR} )}} \\ = \frac{[ I ( {t}_{2} , {λ}_{R} ) - I ( {t}_{1} , {λ}_{R} ) ] I ( {t}_{1} , {λ}_{IR} )}{[ I ( {t}_{2} , {λ}_{IR} ) - I ( {t}_{1} , {λ}_{IR} ) ] I ( {t}_{1} , {λ}_{R} )} \\ = R\end{matrix} \qquad ( 7 )$$

which defines a cluster of points whose slope of y versus x will give R where

x(t)=[I(t₂,λ_{IR})−I(t₁,λ_{IR})]I(t₁,λ_{R})

y(t)=[I(t₂,λ_{R})−I(t₁,λ_{R})]I(t₁,λ_{IR})

y(t)=Rx(t) (8)

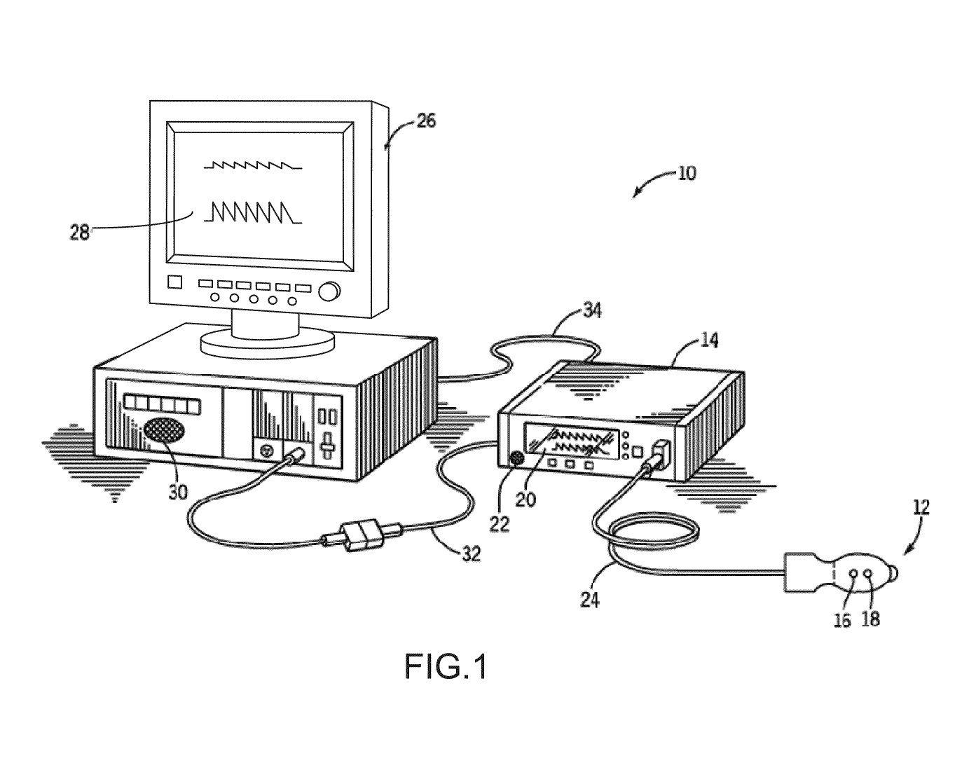

FIG. 1 is a perspective view of an embodiment of a pulse oximetry system 10. System 10 may include a sensor 12 and a pulse oximetry monitor 14. Sensor 12 may include an emitter 16 for emitting light at two or more wavelengths into a patient's tissue. A detector 18 may also be provided in sensor 12 for detecting the light originally from emitter 16 that emanates from the patient's tissue after passing through the tissue.

According to an embodiment and as will be described, system 10 may include a plurality of sensors forming a sensor array in lieu of single sensor 12. Each of the sensors of the sensor array may be a complementary metal oxide semiconductor (CMOS) sensor. Alternatively, each sensor of the array may be a charged coupled device (CCD) sensor. In an embodiment, the sensor array may be made up of a combination of CMOS and CCD sensors. The CCD sensor may comprise a photoactive region and a transmission region for receiving and transmitting data whereas the CMOS sensor may be made up of an integrated circuit having an array of pixel sensors. Each pixel may have a photodetector and an active amplifier.

According to an embodiment, emitter 16 and detector 18 may be on opposite sides of a digit such as a finger or toe, in which case the light that is emanating from the tissue has passed completely through the digit. In an embodiment, emitter 16 and detector 18 may be arranged so that light from emitter 16 penetrates the tissue and is reflected by the tissue into detector 18, such as a sensor designed to obtain pulse oximetry data from a patient's forehead.

In an embodiment, the sensor or sensor array may be connected to and draw its power from monitor 14 as shown. In an embodiment, the sensor may be wirelessly connected to monitor 14 and include its own battery or similar power supply (not shown). Monitor 14 may be configured to calculate physiological parameters based at least in part on data received from sensor 12 relating to light emission and detection. In an alternative embodiment, the calculations may be performed on the monitoring device itself and the result of the oximetry reading may be passed to monitor 14. Further, monitor 14 may include a display 20 configured to display the physiological parameters or other information about the system. In the embodiment shown, monitor 14 may also include a speaker 22 to provide an audible sound that may be used in various other embodiments, such as for example, sounding an audible alarm in the event that a patient's physiological parameters are not within a predefined normal range. Other sensory alarms (e.g. visual, tactile) might also or alternatively be used.

In an embodiment, sensor 12, or the sensor array, may be communicatively coupled to monitor 14 via a cable 24. However, in other embodiments, a wireless transmission device (not shown) or the like may be used instead of or in addition to cable 24.

In the illustrated embodiment, pulse oximetry system 10 may also include a multi-parameter patient monitor 26. The monitor may incorporate a display 28 such as a cathode ray tube type, a flat panel display (as shown) such as a liquid crystal display (LCD) or a plasma display, or any other type of display. Multi-parameter patient monitor 26 may be configured to calculate physiological parameters and to display information from monitor 14 and from other medical monitoring devices or systems (not shown). For example, multiparameter patient monitor 26 may be configured to display an estimate of a patient's blood oxygen saturation generated by pulse oximetry monitor 14 (referred to as an “SpO₂” measurement), as well as other parameters such as pulse rate information from monitor 14, blood pressure from a blood pressure monitor (not shown) and brain state information from an EEG monitor (not shown) on display 28.

Monitor 14 may be communicatively coupled to multi-parameter patient monitor 26 via a cable 32 or 34 that is coupled to a sensor input port or a digital communications port, respectively and/or may communicate wirelessly (not shown). In addition, monitor 14 and/or multi-parameter patient monitor 26 may be coupled to a network to enable the sharing of information with servers or other workstations (not shown). Monitor 14 may be powered by, for example, a battery (not shown) or by an alternative power source such as a wall outlet.

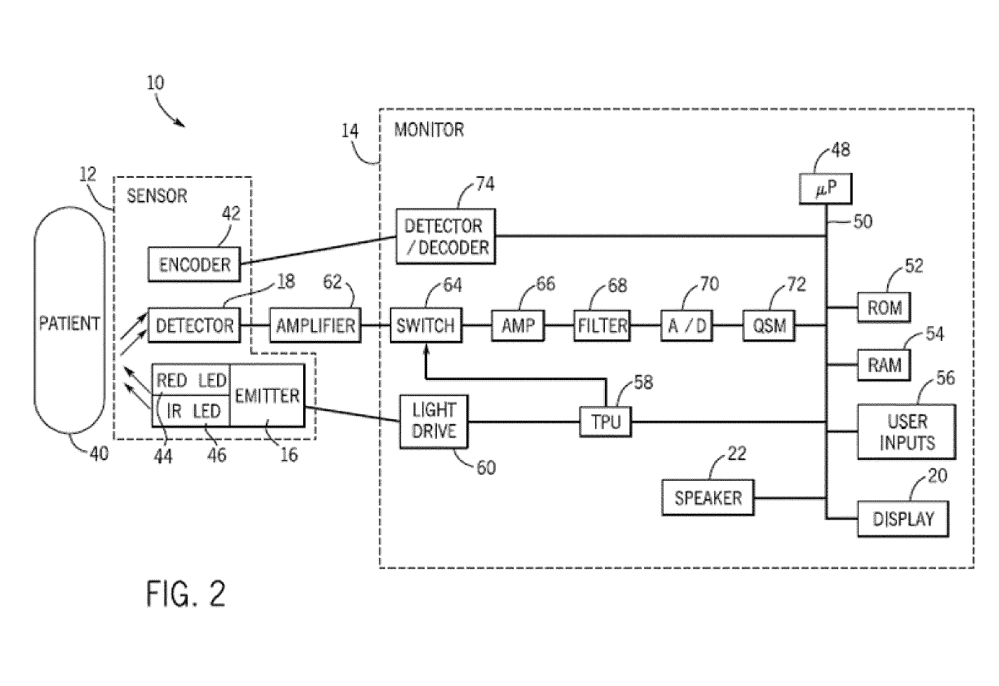

FIG. 2 is a block diagram of a pulse oximetry system, such as pulse oximetry system 10 of FIG. 1, which may be coupled to a patient 40 in accordance with an embodiment. Certain illustrative components of sensor 12 and monitor 14 are illustrated in FIG. 2. Sensor 12 may include emitter 16, detector 18, and encoder 42. In the embodiment shown, emitter 16 may be configured to emit at least two wavelengths of light (e.g., RED and IR) into a patient's tissue 40. Hence, emitter 16 may include a RED light emitting light source such as RED light emitting diode (LED) 44 and an IR light emitting light source such as IR LED 46 for emitting light into the patient's tissue 40 at the wavelengths used to calculate the patient's physiological parameters. In one embodiment, the RED wavelength may be between about 600 nm and about 700 nm, and the IR wavelength may be between about 800 nm and about 1000 nm. In embodiments where a sensor array is used in place of single sensor, each sensor may be configured to emit a single wavelength. For example, a first sensor emits only a RED light while a second only emits an IR light.

It will be understood that, as used herein, the term “light” may refer to energy produced by radiative sources and may include one or more of ultrasound, radio, microwave, millimeter wave, infrared, visible, ultraviolet, gamma ray or X-ray electromagnetic radiation. As used herein, light may also include any wavelength within the radio, microwave, infrared, visible, ultraviolet, or X-ray spectra, and that any suitable wavelength of electromagnetic radiation may be appropriate for use with the present techniques. Detector 18 may be chosen to be specifically sensitive to the chosen targeted energy spectrum of the emitter 16.

In an embodiment, detector 18 may be configured to detect the intensity of light at the RED and IR wavelengths. Alternatively, each sensor in the array may be configured to detect an intensity of a single wavelength. In operation, light may enter detector 18 after passing through the patient's tissue 40. Detector 18 may convert the intensity of the received light into an electrical signal. The light intensity is directly related to the absorbance and/or reflectance of light in the tissue 40. That is, when more light at a certain wavelength is absorbed or reflected, less light of that wavelength is received from the tissue by the detector 18. After converting the received light to an electrical signal, detector 18 may send the signal to monitor 14, where physiological parameters may be calculated based on the absorption of the RED and IR wavelengths in the patient's tissue 40.

In an embodiment, encoder 42 may contain information about sensor 12, such as what type of sensor it is (e.g., whether the sensor is intended for placement on a forehead or digit) and the wavelengths of light emitted by emitter 16. This information may be used by monitor 14 to select appropriate algorithms, lookup tables and/or calibration coefficients stored in monitor 14 for calculating the patient's physiological parameters. Encoder 42 may, for instance, be a coded resistor which stores values corresponding to the type of sensor 12 or the type of each sensor in the sensor array, the wavelengths of light emitted by emitter 16 on each sensor of the sensor array, and/or the patient's characteristics. In another embodiment, encoder 42 may include a memory on which one or more of the following information may be stored for communication to monitor 14; the type of the sensor 12; the wavelengths of light emitted by emitter 16; the particular wavelength each sensor in the sensor array is monitoring; a signal threshold for each sensor in the sensor array; any other suitable information; or any combination thereof.

Encoder 42 may also contain information specific to patient 40, such as, for example, the patient's age, weight, and diagnosis. The information specific to patient 40 may be the patient data collected using one or more sensors. For example, encoder 42 may contain information related to heart rate (HR), respiratory rate, blood pressure (BP—Mean Arterial Pressure (MAP), Systolic Pressure, Diastolic Pressure), as well as derived hemodynamic parameters (ratios, product or differences of heart rate and the components of BP e.g., Systolic/Diastolic or MAP/HR), Bispectral Index™ (BIS™), SpO2, temperature, ScO2, rSO2, etc.) and information about patient interventions (e.g., the start of a surgical procedure, intubation of the patient, the administration of drugs, etc.). This information may allow monitor 14 to determine, for example, patient-specific threshold ranges in which the patient's physiological parameter measurements should fall and to enable or disable additional physiological parameter algorithms.

In an embodiment, signals from detector 18 and encoder 42 may be transmitted to monitor 14. In the embodiment shown, monitor 14 may include a general-purpose microprocessor 48 connected to an internal bus 50. Microprocessor 48 may be adapted to execute software, which may include an operating system and one or more applications, as part of performing the functions described herein. Also connected to bus 50 may be a read-only memory (ROM) 52, a random access memory (RAM) 54, user inputs 56, display 20, and speaker 22.

RAM 54 and ROM 52 are illustrated by way of example, and not limitation. Any suitable computer-readable media may be used in the system for data storage. Computer-readable media are capable of storing information that can be interpreted by microprocessor 48. This information may be data or may take the form of computer-executable instructions, such as software applications, that cause the microprocessor to perform certain functions and/or computer-implemented methods. Depending on the embodiment, such computer-readable media may include computer storage media and communication media. Computer storage media may include volatile and non-volatile, removable and non-removable media implemented in any method or technology for storage of information such as computer-readable instructions, data structures, program modules or other data. Computer storage media may include, but is not limited to, RAM, ROM, EPROM, EEPROM, flash memory or other solid state memory technology, CD-ROM, DVD, or other optical storage, magnetic cassettes, magnetic tape, magnetic disk storage or other magnetic storage devices, or any other medium which can be used to store the desired information and which can be accessed by components of the system.

In the embodiment shown, a time processing unit (TPU) 58 may provide timing control signals to a light drive circuitry 60, which may control when emitter 16 is illuminated and multiplexed timing for the RED LED 44 and the IR LED 46. TPU 58 may also control the gating-in of signals from detector 18 through an amplifier 62 and a switching circuit 64. These signals are sampled at the proper time, depending upon which light source is illuminated. The received signal from detector 18 may be passed through an amplifier 66, a low pass filter 68, and an analog-to-digital converter 70. The digital data may then be stored in a queued serial module (QSM) 72 (or buffer) for later downloading to RAM 54 as QSM 72 fills up. In one embodiment, there may be multiple separate parallel paths having amplifier 66, filter 68, and A/D converter 70 for multiple light wavelengths or spectra received.

In an embodiment, microprocessor 48 may determine the patient's physiological parameters, such as SpO₂ and pulse rate, using various algorithms and/or look-up tables based on the value of the received signals and/or data corresponding to the light received by detector 18. Signals corresponding to information about patient 40, and particularly about the intensity of light emanating from a patient's tissue over time, may be transmitted from encoder 42 to a decoder 74. These signals may include, for example, encoded information relating to patient characteristics. Decoder 74 may translate these signals to enable the microprocessor to determine the thresholds based on algorithms or look-up tables stored in ROM 52. User inputs 56 may be used to enter information about the patient, such as age, weight, height, diagnosis, medications, treatments, and so forth. In an embodiment, display 20 may exhibit a list of values which may generally apply to the patient, such as, for example, age ranges or medication families, which the user may select using user inputs 56.

Microprocessor 48 may also track changes in patient physiological data and/or physiological parameters over time and may calculate additional physiological parameters and/or statistics. For example, microprocessor 48 may track variability of patient parameters. Variability can be assessed over a specific time period (e.g., one or five minutes) and may be quantified by various well-known techniques, such as a variance or standard deviation, the range (the maximum minus the minimum over the specified time period) or the interquartile range (the 75^{th} percentile minus the 25^{th} percentile). For non-normally distributed quantities, variability may be quantified by the number of excursions above or below a threshold during the specified time period or the area between the time trend of the signal and the threshold. For example, in an embodiment, the variability of SpO2 may be quantified by the number of excursions below a threshold of 90 or by integrating the area between the SpO2 trend and a line representing the threshold value of 90.

The optical signal through the tissue can be degraded by noise, among other sources. One source of noise is ambient light that reaches the light detector. Another source of noise is electromagnetic coupling from other electronic instruments. Movement of the patient may introduce noise and affect the signal. For example, the contact between the detector and the skin, or the emitter and the skin, can be temporarily disrupted when movement causes either to move away from the skin. In addition, because blood is a fluid, it responds differently than the surrounding tissue to inertial effects, thus resulting in momentary changes in volume at the point to which the oximeter probe is attached.

Noise (e.g., from patient movement) can degrade a pulse oximetry signal relied upon by a physician, without the physician's awareness. This is especially true if the monitoring of the patient is remote, the motion is too small to be observed, or the doctor is watching the instrument or other parts of the patient, and not the sensor site. Processing pulse oximetry (i.e., PPG) signals may involve operations that reduce the amount of noise present in the signals or otherwise identify noise components in order to prevent them from affecting measurements of physiological parameters derived from the PPG signals.

In certain embodiments it is useful to change or limit filtering of a pulse oximetry signal or remove it altogether in order to track changes in various parameters of the signal. By limiting or restricting filtering, small changes, for example in the variability of a pulse oximetry signal, which may represent important physiological information, may be preserved. Further, in an embodiment, two or more versions of a pulse oximetry signal may be displayed to a clinician: one being filtered for noise and others being filtered differently or not at all in order to track various parameters of the pulse oximetry signal. In the latter case, more data may be preserved by limiting, changing or eliminating the filtering of the signal.

It will be understood that the disclosure is applicable to any suitable signals and that PPG signals are used merely for illustrative purposes. Those skilled in the art will recognize wide applicability to other signals including, but not limited to other biosignals (e.g., electrocardiogram, electroencephalogram, electrogastrogram, electromyogram, heart rate signals, pathological sounds, ultrasound, plethysmogram, or any other suitable biosignal).

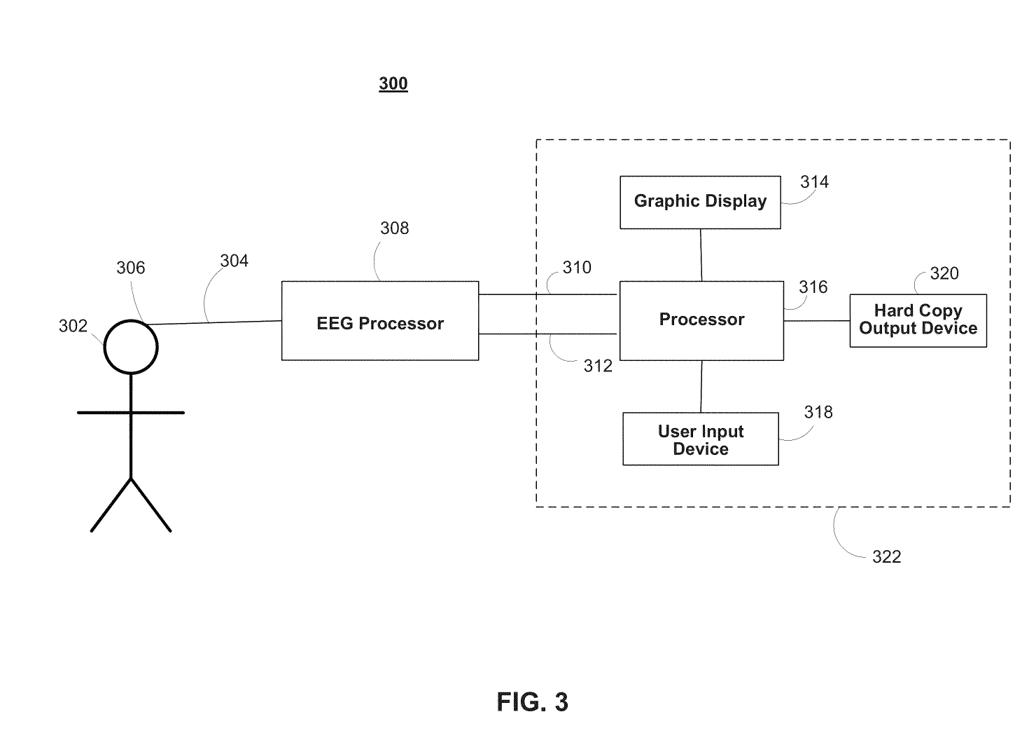

EEG data acquisition apparatus may also be used with the monitoring system for measuring and collecting physiological data related to a patient's brain state. FIG. 3 shows a schematic view of system for detecting brain state in accordance with an embodiment. The EEG data acquisition apparatus 300 provides an input signal over cable 304 to an EEG processing system 308. The EEG processing system may be the same or different than the processor used in system 10 of FIG. 1. Said EEG processing system 308 in turn provides an input signal 310 to a monitoring system 322 which monitors a patient's sedative or hypnotic state, or a patient's analgesic state and analgesic adequacy. The input signal 304 may be, for example, an EEG signal generated in known fashion by one or more EEG electrodes 306, or alternatively, by an amplifier or other known EEG processing components. The EEG leads are connected to a patient's head 302 by a set of one or more surface electrodes 306. In an embodiment, surface electrodes 306 are part of a BIS™ Sensor. The EEG signals are detected by the electrodes 306 and transmitted over a cable 304 to the EEG processing system 308. The input signal 304 generated by one or more EEG electrodes 306 may be applied to any device used to process EEG signals (e.g., such as a Bispectral Index generator of the type disclosed in U.S. Pat. No. 5,458,117, which is incorporated herein by reference in its entirety). The EEG processing device 308 generates a first output signal 310 which is representative of the cerebral activity of the patient. In an embodiment, the output signal 310 is representative of the patient's sedative or hypnotic state.

The EEG processing device 308 generates a second output signal 312 which is representative of the electromyographic (EMG) activity of the patient. In an embodiment, the second output signal 312 is representative of the level of muscle activity or tone in the muscles in the region immediately beneath the electrodes 306. Monitoring system 322 receives the first output signal 310 representative of cerebral activity of a patient and the second output signal 312 representative of the EMG activity of the patient and may compute from one or both of the two signals an index representative of the analgesic adequacy and analgesic state of the patient. This index may be displayed on the graphics display 314 which is connected to the processor 316. Processor 316 may be the same or separate from EEG processor 308. Printed output of the index may also be available on the hard copy output device 320 which is connected to the processor 316. The operator may interact with the acquisition and analysis components of the system by means of a user input device 318 with feedback on the graphics display 314. In an embodiment, first output signal 310, which is representative of the cerebral activity of the patient, is the Bispectral Index™ (BIS™), as generated by the product line of level of consciousness monitors sold by Nellcor Puritan Bennett, LLC such as the A2000™ monitor, the BIS Vista™ monitor, or the BISx™ module used in conjunction with a third-party patient monitoring system.

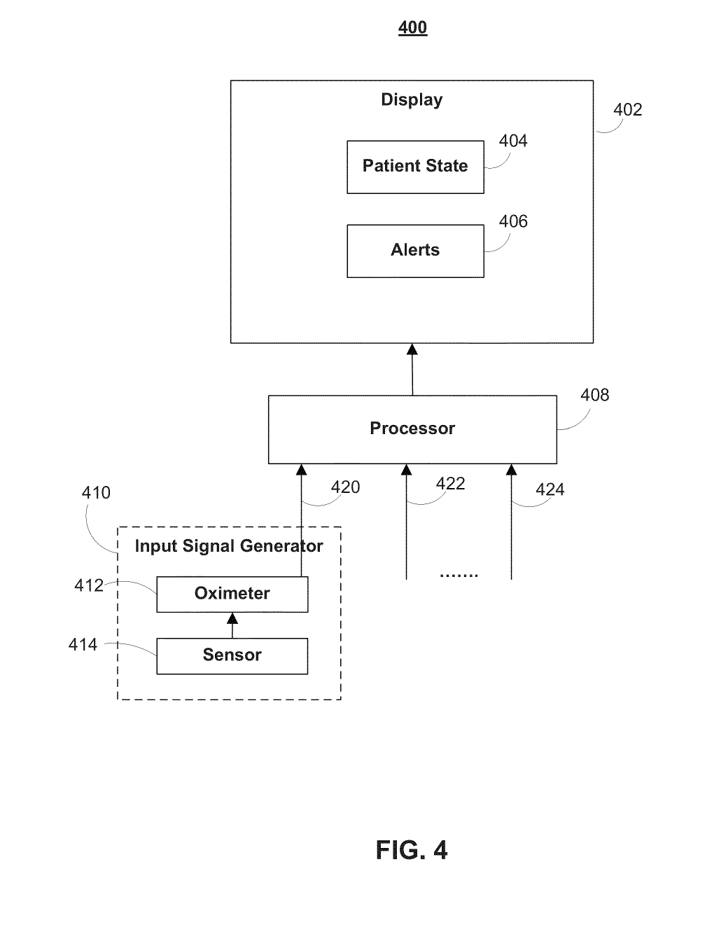

FIG. 4 is an illustrative block diagram of a patient monitoring system 400 capable of monitoring a patient. For example, patient monitoring system 400 may be used to monitor a patient during a surgical procedure. System 400 includes a display 402 and a plurality of inputs 420, 422 and 424. While FIG. 4 shows 3 inputs into monitoring system 400, it will be understood by those of skill in the art that monitoring system 400 may include more or less inputs as necessary. System 400 also includes processor 408 used to process inputs 420, 422 and 424 in order to generate patient state information 404 and alerts 406. Inputs 420, 422 and 424 may be provided from any suitable data source, data generating source, data input source, data generating equipment, physiological sensor or any combination thereof.

For example, input 420 may be provided from pulse oximetry sensor system, such as the pulse oximetry sensor system 10 of FIGS. 1 and 2, having an input signal generator 410. In an embodiment, the input signal generator 410 generates an input signal 420. As illustrated, input signal generator may include oximeter 412 coupled to sensor 414, the output of which may be provided as input signal 420. It will be understood that input signal generator 410 may include any suitable signal source, signal generating data, signal generating equipment, or any combination thereof to produce input 420. Signals 420, 422 and 424 may be any suitable signal or signals, such as, for example, biosignals (e.g., electrocardiogram, electroencephalogram, electrogastrogram, electromyogram, heart rate signals, pathological sounds, ultrasound, plethysmogram, photoplethysmogram, or any other suitable biosignal).

In an embodiment, signal 420, 422 and 424 may be coupled to processor 408. Processor 408 may be any suitable software, firmware, and/or hardware, and/or combinations thereof for processing signals 420, 422 and 424. For example, processor 408 may include one or more hardware processors (e.g., integrated circuits), one or more software modules, computer-readable media such as memory, firmware, or any combination thereof. Processor 408 may, for example, be a computer or may be one or more chips (i.e., integrated circuits). Processor 408 may perform the calculations associated with continuous wavelet transforms as well as the calculations associated with any suitable interrogations of the transforms. Processor 408 may perform any suitable signal processing of signals 420, 422 and 424 to filter signals 420, 422 and 424, such as any suitable band-pass filtering, adaptive filtering, closed-loop filtering, and/or any other suitable filtering, and/or any combination thereof.

Processor 408 may be coupled to one or more memory devices (not shown) or incorporate one or more memory devices such as any suitable volatile memory device (e.g., RAM, registers, etc.), non-volatile memory device (e.g., ROM, EPROM, magnetic storage device, optical storage device, flash memory, etc.), or both. The memory may be used by processor 408 to, for example, store data corresponding to a continuous wavelet transform of input signal 420, such as data representing a scalogram. In one embodiment, data representing a scalogram may be stored in RAM or memory internal to processor 408 as any suitable three-dimensional data structure such as a three-dimensional array that represents the scalogram as energy levels in a time-scale plane. Any other suitable data structure may be used to store data representing a scalogram.

Processor 408 may perform the calculations of physiological parameters based at least in part on the physiological data collected from the sensors at inputs 420, 422 and 424. Processor 408 may also classify the patient as being in one of a plurality of patient states based on at least one of the calculated physiological parameters. Additionally, processor 408 may provide alerts and data to display 402 in order to display the physiological data, physiological parameters and patient state classification.

It will be understood that systems 10 (FIGS. 1 and 2) and 300 (FIG. 3) or any other suitable sensor or sensor system may be incorporated into system 400, or connected via one or more of inputs 420, 422 and 424. Input signal generator 410 may be implemented as parts of sensor 12 and monitor 14 and processor 408 may be implemented as part of monitor 14. EEG data acquisition apparatus 300 may be connected at any of inputs 420, 422 and 424 or integrated with processor 408. Similarly, a blood pressure monitor, heart rate monitor, or any other suitable physiological sensor or data input source may be connected to any of inputs 420, 422 and 424 or other suitable inputs as necessary.

In certain embodiments, data may be input into the system 400 using a keyboard, mouse, internet connection, automatic download or any other suitable method for inputting data known to those of skill in the art. Inputs 420, 422 and 424 may also provide data associated with any suitable signal or signals, such as, for example, biosignals (e.g., electrocardiogram, electroencephalogram, electrogastrogram, electromyogram, heart rate signals, pathological sounds, ultrasound, plethysmogram, photoplethysmogram, or any other suitable biosignal).

Processor 408 may be coupled to display 402. Display 402 may be incorporated into a monitor such as monitor 14 or 26 (FIG. 1) or graphic display 314 (FIG. 2). Alternatively, or in addition to display 402, processor 408 may be coupled to any suitable output device such as, for example, one or more medical devices (e.g., a medical monitor that displays various physiological parameters, a medical alarm, or any other suitable medical device that either displays physiological parameters or uses the output of processor 408 as an input), one or more display devices (e.g., monitor, PDA, mobile phone, any other suitable display device, or any combination thereof), one or more audio devices, one or more memory devices (e.g., hard disk drive, flash memory, RAM, optical disk, any other suitable memory device, or any combination thereof), one or more printing devices, any other suitable output device, or any combination thereof.

For ease of illustration, system 400 is shown as having three inputs, inputs 420, 422 and 424. It will be understood that any suitable number of inputs may be used. Inputs 420, 422 and 424 may receive patient clinical information including, for example, measured physiological parameters from the patient (e.g., heart rate (HR), respiratory rate, blood pressure (BP—Mean Arterial Pressure (MAP), Systolic Pressure, Diastolic Pressure), as well as derived hemodynamic parameters (ratios, products or differences of heart rate and the components of BP e.g., Systolic/Diastolic or MAP/HR), Bispectral Index™ (BIS™), SpO2, temperature, ScO2, rSO2, etc.) and information about patient interventions (e.g., the start of a surgical procedure, intubation of the patient, the administration of drugs, etc.). This information may be provided to inputs 420, 422 and 424 directly from one or more medical devices or sensors, may be accessed from one or more databases, or may be input by a user.

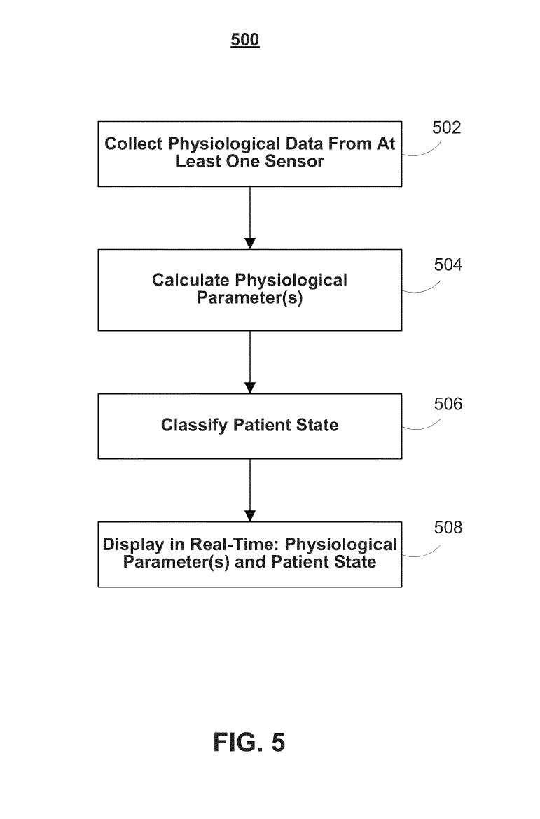

FIG. 5 is a flow chart 500 of illustrative steps involved in calculating physiological parameters based on physiological data from a patient, classifying a patient's state and displaying the information. The information may be displayed in real-time or at another clinically desired interval. The steps of flow chart 500 may be carried out, for example, using patient monitoring system 400 of FIG. 4. At step 502, physiological data is collected from at least one sensor or sensor system, for example, using inputs 420, 422 and 424 (FIG. 4). The physiological data may include brain state data from an EEG monitor system (such as EEG data acquisition apparatus 300 of FIG. 3), mean arterial pressure (MAP) data from a blood pressure monitor, oxygen saturation data from an oximeter (such as oximeter system 10 of FIGS. 1 and 2), measures of hemodynamic state, cardiovascular function such as heart rate, diastolic pressure, systolic pressure, stroke volume, cardiac output and flow, other brain monitoring measurements as well as other measures of patient brain state, measures of analgesic adequacy, or any other suitable physiological data. The physiological data may correspond to the physiological data and/or physiological parameters that the physician is monitoring during a surgical procedure. In an embodiment, only selected physiological parameters are provided to patient monitoring system 400 to classify a patient's state. In another embodiment, physiological data from multiple sensors are provided to patient monitoring system 400 and only selected physiological data and/or physiological parameters are monitored and used to classify a patient's state. The physiological data may be collected in step 502 by processor 408 and may be stored in memory, such as the memory described with respect to monitor 14 (FIG. 2). In certain embodiments, the physiological data may be filtered, for example using a median or trim-mean filter, or any other suitable filtering means, to eliminate artifact, over a particular time period to provide an estimate for that time period. It will be understood that any suitable number of inputs may be used. In an embodiment inputs 420, 422 and 424 may receive patient characteristics including, for example, a patient's medical history, surgical history, demographic information (e.g., age, sex, weight, body mass index (BMI), etc.). Inputs 420, 422 and 424 may also receive population characteristics, for example, data from a patient population database. The population characteristics may include information about a reference population. The reference population may include a data set of patient characteristics and patient clinical information for a set of patients.

At step 504, the processor 408 calculates at least one physiological parameter based at least in part on the physiological data collected in step 502. The one or more parameters calculated in step 508 may include performing statistical operations (e.g., variance, range (maximum-minimum over a particular time period), average, standard deviation) on the physiological parameters (e.g., SpO2, BIS, MAP, MAC, finger pressure, Saturation Pattern Detection Index (SPDi), plethysmogram or photoplethysmogram, PVI, cerebral or somatic oximetry methodologies (e.g., ScO2 and rSO2, respectively) or any other suitable calculated parameter. The calculated parameters may be updated at a particular frequency (e.g., every second, minute, 10 minutes etc.). The one or more parameters calculated in step 504 can be combined and used to make a determination of whether a patient is in an undesirable state (e.g., a state related to hypoperfusion). In an embodiment, at step 504 the processor 408 may compare one or more calculated parameters, or physiological data from step 502, to a respective parameter threshold. For example, the current value of a calculated parameter in a patient may be higher than, lower than, or equal to a reference state for that parameter. In this example, higher than, lower than, and equal to the reference state are three patient states associated with the physiological parameter.

In an embodiment, population-based norms may be used to define patient states. For example, a reference set for a monitored physiological parameter may be associated with a mean value or mean range of values for the parameter calculated from a patient population database. The patient state may be defined based on where the patient falls, e.g., higher than, lower than, or equal to the reference state. Multiple parameters, calculated in step 504, may be used to compare to population-based norms to determine a patient's state. In an embodiment, the patient states may be adjusted from the population-based characteristics based on patient characteristics (e.g., age). For example, a patient's BIS or SpO2 value at a particular point in time may be compared to a threshold, such as a population average. In certain embodiments, parameter values immediately following a period of greater than a pre-set time period (e.g. 15 minutes) since the last value of that parameter was updated (e.g., infrequent non-invasive MAP assessments) are declared missing. Classification of patient state and estimation of relative risk may not be provided for periods with missing data.

In step 506 the processor 408 classifies the patient as being in one of a plurality of states based on at least one of the physiological parameters calculated in step 504. The classification of the patient's state may be performed by processor 408 and updated at a particular frequency. For example, in an embodiment the processor 408 classifies the patient each minute as being “BIS-HI” or “BIS-LO”, based on whether the patient's BIS value for that minute is greater than 45 (BIS-HI) or less than or equal to 45 (BIS-LO). In an embodiment, the processor 408 classifies the patient each minute being “MAP-HI” or “MAP-LO”, based on whether the patient's MAP value for that minute is greater than 75 (MAP-HI) or less than or equal to 75 (MAP-LO). In an embodiment the processor 408 classifies the patient each minute being “SpO2 Range-HI” or “SpO2 Range-LO”, based on whether the patient's SpO2 Range value for the immediately preceding 5 minutes indicates an SpO2 variance having a range greater than 2 (SpO2 Range-HI) or less than or equal to 2 (SpO2 Range-LO). It will be appreciated by those of skilled in the art that the thresholds and time periods used in the above examples may be altered as appropriate.

In certain embodiments, classification of patient state may include determining a patient's condition based at least in part on two or more of the parameters calculated in step 504. For example, in an embodiment a patient's condition may be determined based at least in part on a brain state parameter and an oxygen saturation parameter. In an embodiment, a patient's condition may be determined based at least in part on a brain state parameter and a blood pressure parameter. In an embodiment a patient's condition may be determined based at least in part on an oxygen saturation parameter and a blood pressure parameter. In an embodiment a patient's condition may be determined based at least in part on a brain state parameter, a blood pressure parameter and an oxygen saturation parameter.

For example, if a patient is classified based at least in part on a brain state parameter, a blood pressure parameter and an oxygen saturation parameter, there may be 8 potential states, each of which has a relative risk of an adverse outcome associated with it. These relative risks may be derived from the Cox Proportional Hazards modeling method, or any other suitable modeling method, and may be derived for different outcomes. The most appropriate modeling technique will depend on the structure of the available data and endpoints and includes, in addition to Cox Proportional Hazards modeling, for example: logistic regression, general linear modeling, generalized linear modeling, linear regression and other modeling techniques well known in the art. The Cox Proportional Hazards modeling method, for example, is based on the notion that if the proportional hazards assumption holds (or, is assumed to hold) then it is possible to estimate the effect parameter(s) without any consideration of the hazard function. The Cox Proportional Hazards model may be specialized if a reason exists to assume that the baseline hazard follows a parametric form. The Table below lists an example of the 8 states and provides the relative risk of 90-day postoperative mortality compared to a reference state:

TABLE 1
Characterization of Patient States

1) BIS HI, MAP LO, SpO2 Range LO: Rel. Risk = 1.32
2) BIS HI, MAP HI, SpO2 Range LO: Rel. Risk = 0.67
3) BIS LO, MAP LO, SpO2 Range LO: Rel. Risk = 1.61
4) BIS LO, MAP HI, SpO2 Range LO: Rel. Risk = 0.90
5) BIS HI, MAP LO, SpO2 Range HI: Rel. Risk = 1.57
6) BIS HI, MAP HI, SpO2 Range HI: Rel. Risk = 1.07
7) BIS LO, MAP LO, SpO2 Range HI: Rel. Risk = 2.22
8) BIS LO, MAP HI, SpO2 Range HI: Rel. Risk = 1.39

In certain embodiments, risks other than 90-day postoperative mortality may be calculated. In the example above, the patient is classified each minute as to the state that they are in relative to their 90-day postoperative mortality risk, however one of ordinary skill in the art will appreciate that the patient classification may be updated over a shorter or longer period than one minute.

In step 506 the processor 408 may use the patient states to determine various risk states for a patient. For example, the risk states relative to a reference state of 1.0 may be: elevated risk (e.g., relative risk of 2.22), normal risk (e.g., relative risk of 1.07), or decreased risk (e.g., relative risk of 0.67). Table 1 contains numerical values for the relative risk measures. It should be understood that any other suitable indication of relative risk may be used such as, for example, discreet values or rankings (i.e., high, normal, low). Specific ranges of each of the parameters used to define the eight states in Table 1 are associated with worse outcomes and each may be used to define a “single risk” state. Outcomes in states associated with BIS LO (3, 4, 7 and 8) are worse than outcomes in states associated with BIS HI (1, 2, 5 and 6). BIS LO is thus a single risk state associated with worse outcome. Similarly, outcomes in states associated with MAP LO (1, 3, 5 and 7) are worse than outcomes in states associated with MAP HI (2, 4, 6 and 8). MAP LO is thus a single risk state associated with worse outcome. Specific combinations of parameters may also be used to define “double risk” states. In the embodiment shown in Table 1, the combination of BIS LO and MAP LO (states 5 and 7) comprises such a double risk state. Additional risk is incurred in these two states when the patient is classified as Hi SpO2 Range, which is a “triple risk” state (state 7). Single, double and triple risk states may each be used to classify patients into states associated with specific relative risks, depending on the patient data available. Similarly, any number of physiological parameters may be used in combination to classify the patient's state. Also, the system may classify the patient as being in a reduced risk state. In the embodiment shown in the table above, the reduced risk state occurs when a patient is in one of the following two states, state 2: BIS HI, MAP HI, SpO2 Range LO; and state 4: BIS LO, MAP HI, SpO2 Range LO. In an embodiment, the system may classify the patient as being in a normal risk state when the patient is not in an elevated or reduced risk state.

The Cox Proportional Hazards Model technique may be used to derive the relative risk associated with specific amounts of time in each state. For example, the relative risk associated with each state may be calculated per minute of time in that state. One embodiment calculates the cumulative time a patient spends in each state and continuously calculates the cumulative risk that a patient has experienced from the beginning of a calculation period until the present.

In step 508, one or more of the physiological data, physiological parameters, patient state and patient condition information may be displayed. The information may be displayed, for example, on display 402 of system 400. The system 400 may display the patient's instantaneous state classification to a clinician caring for the patient by means of display 402, a warning light, an audible or visual alarm, or any other suitable communications means. The system 400 may also transmit the patient's instantaneous state classification to a clinician or other health care personnel using wireless communications means such as a pager, a text message or an e-mail. The system 400 may also transmit the patient's instantaneous state classification to an anesthesia or medical information system for remote monitoring and data recording. For example, patient state information 404 in display 402 may indicate that the patient is in a low BIS value state. During step 508, the system 400 may also display the cumulative time that a patient spends in one or more particular patient states. In an embodiment the relative risk associated with the patient's state (e.g., elevated, normal, reduced) may be displayed instead of or in addition to the current patient state information. For example, the patient state information 404 in display 402 may also indicate that a low BIS value state is associated with an increased risk of mortality. In an embodiment, the system 400 may make use of processing windows and alarm latencies to enhance the stability of the state assessment. For example, a patient's state might be calculated using the BIS, MAP or SpO2 Range averaged over a recent time period, (e.g. 5 minutes). System 400 may also display information on patient states from previous time periods in order to illustrate a progression of the patient's state over time. System 400 may also display information on one or more patient states that are associated with a relatively lower risk than the current state and/or the amount of change in one or more physiological parameters that may result in a change in patient state.

At step 508, patient monitoring system 400 may also generate and provide one or more alerts when the patient is in a particular patient state, such as an undesirable patient state. The alert may be audible, visual, tactile or any other suitable alert. In some embodiments, patient monitoring system 400 may output the current patient state, the current risk assessment associated with a given endpoint, and alerts based on time spent in a particular state.

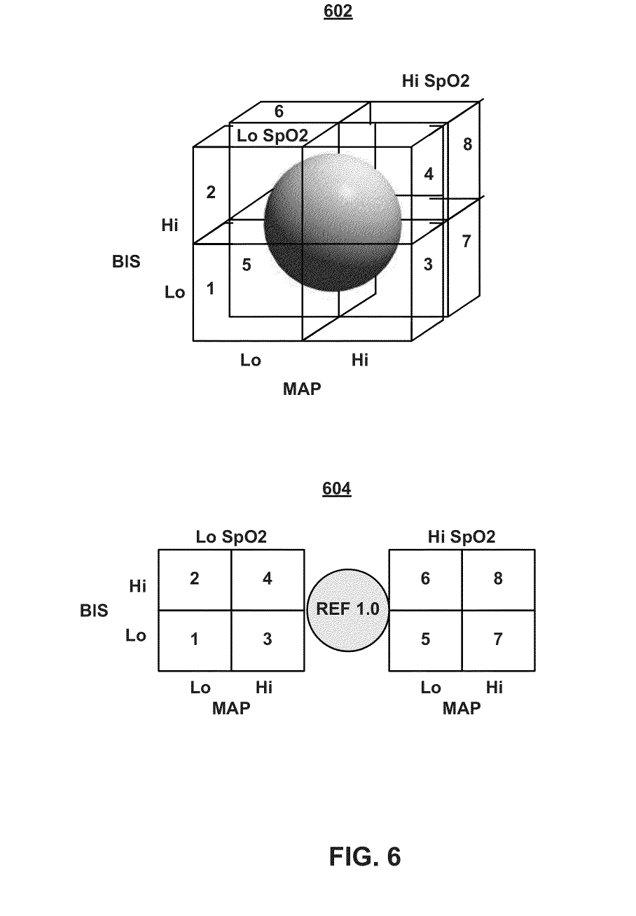

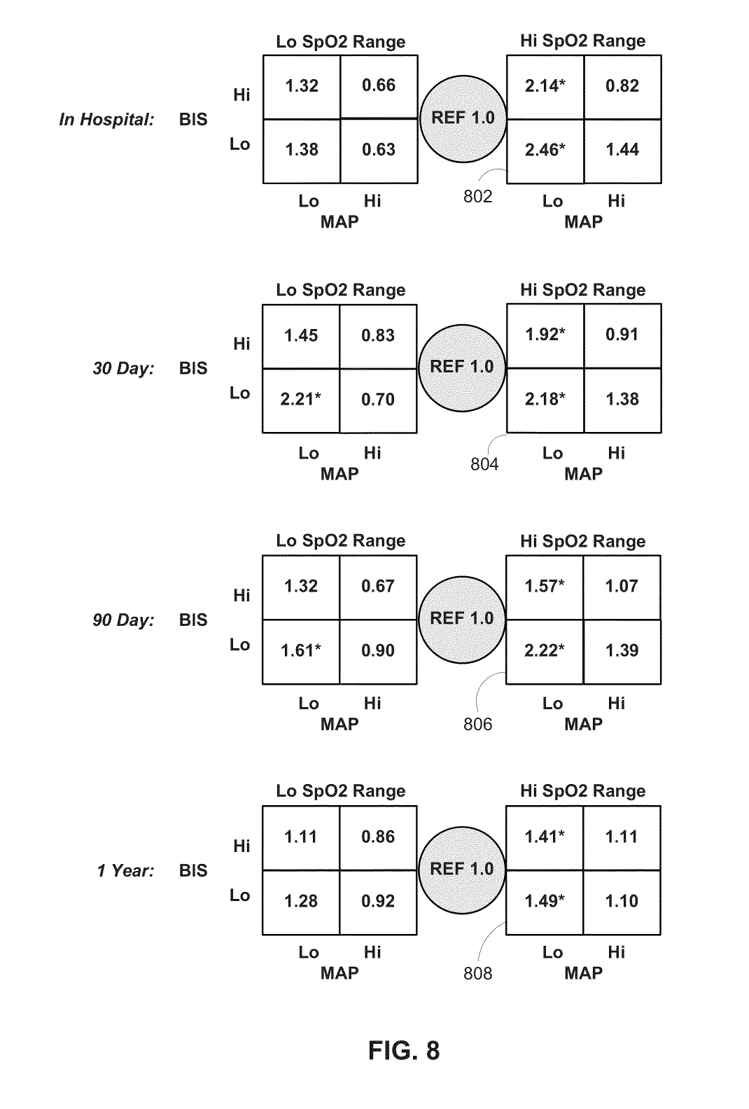

FIG. 6 shows illustrative displays of patient state and risk assessment information that may be displayed, for example, in display 402 of patient monitoring system 400 (FIG. 4). In an embodiment, the patient risk assessment information may be shown in a three-dimensional grid or space, e.g., in a 2×2×2 cube (8 cells+a central reference cell) as shown in display 602. The patient risk assessment information may also be shown in a two-dimensional grid space, where the third-dimension of plot 602 is separated into a flattened two-dimensional space (8 cells+a ninth reference cell) as shown in display 604. The reference cell in displays 602 and 604 is depicted as a sphere; however it could also be displayed as a cube, or any other suitable shape. Displays 602 and 604 will be described in more detail with reference to the illustrative example shown in FIG. 8.

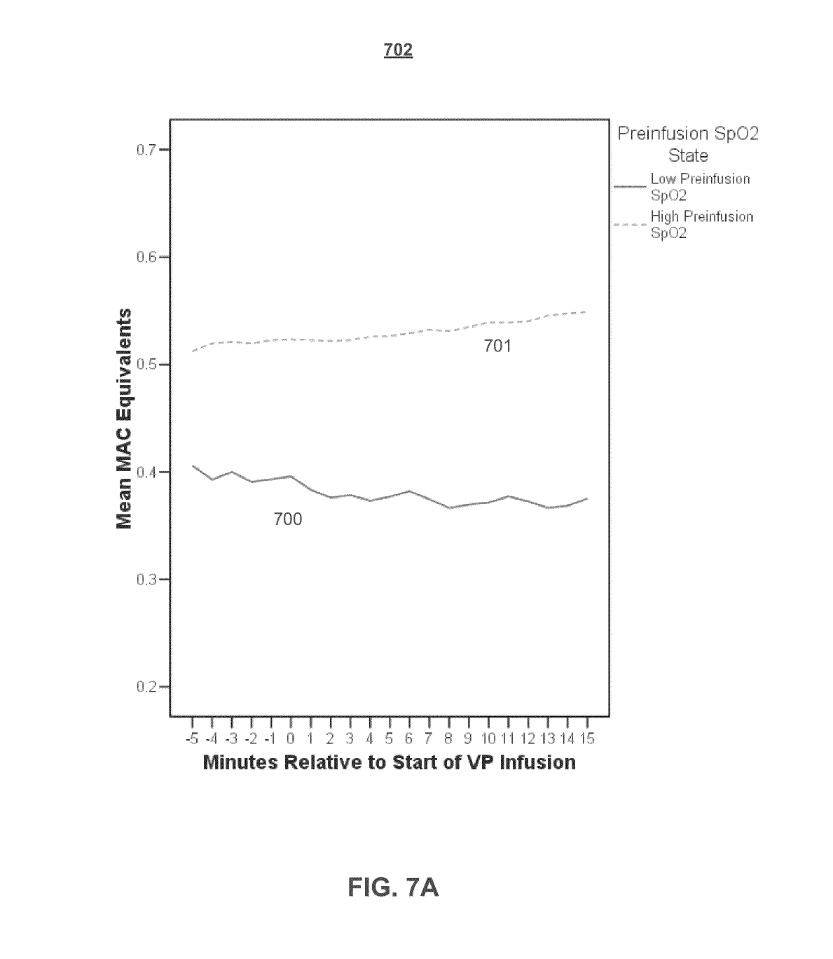

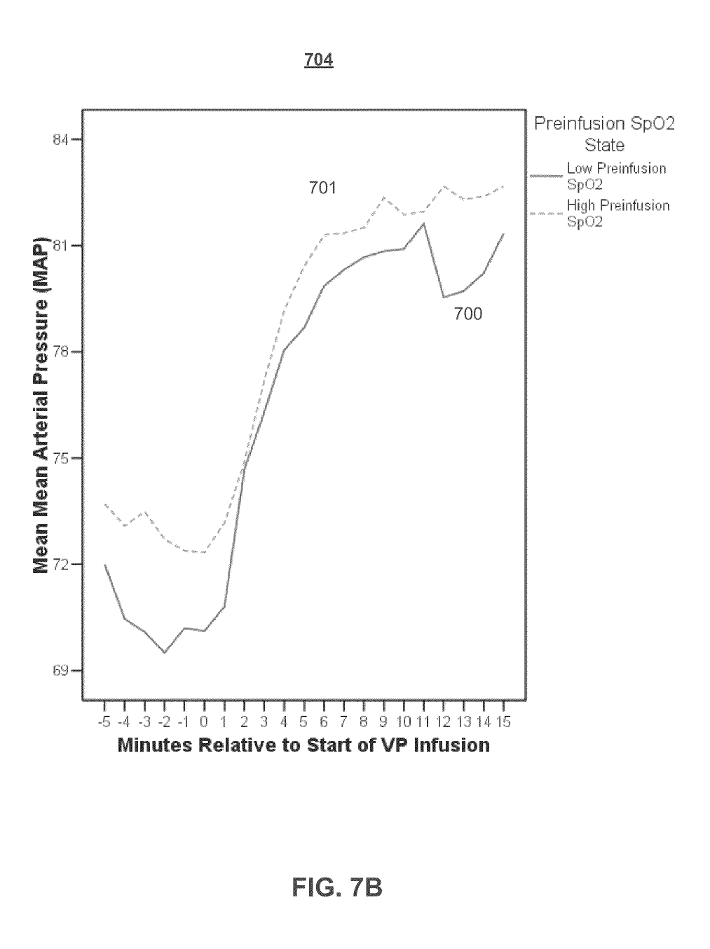

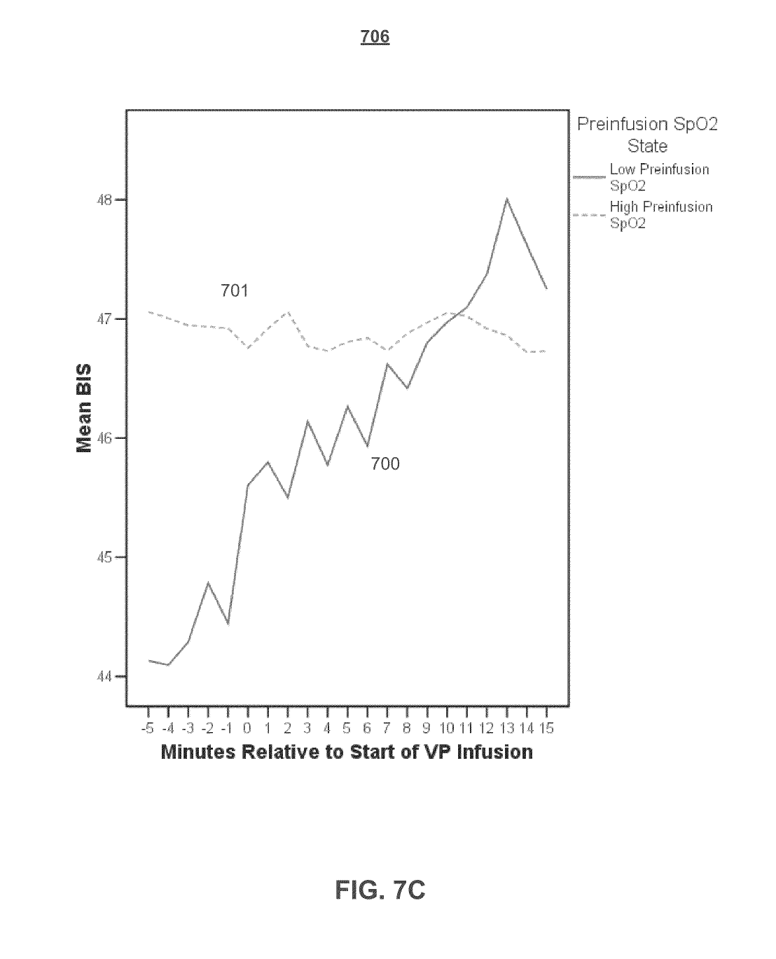

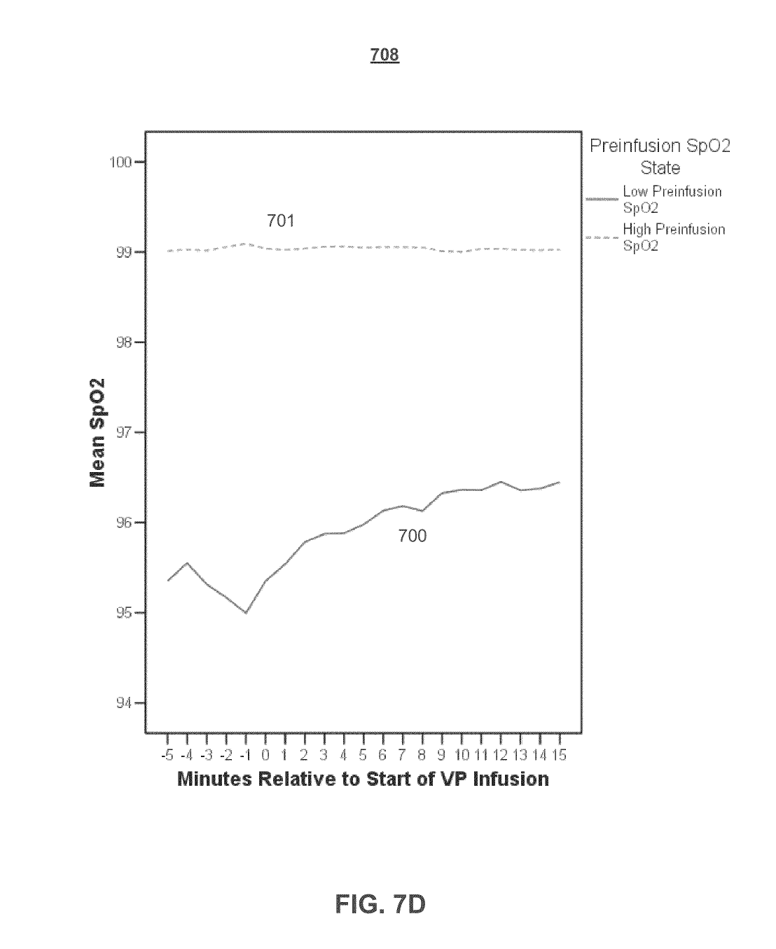

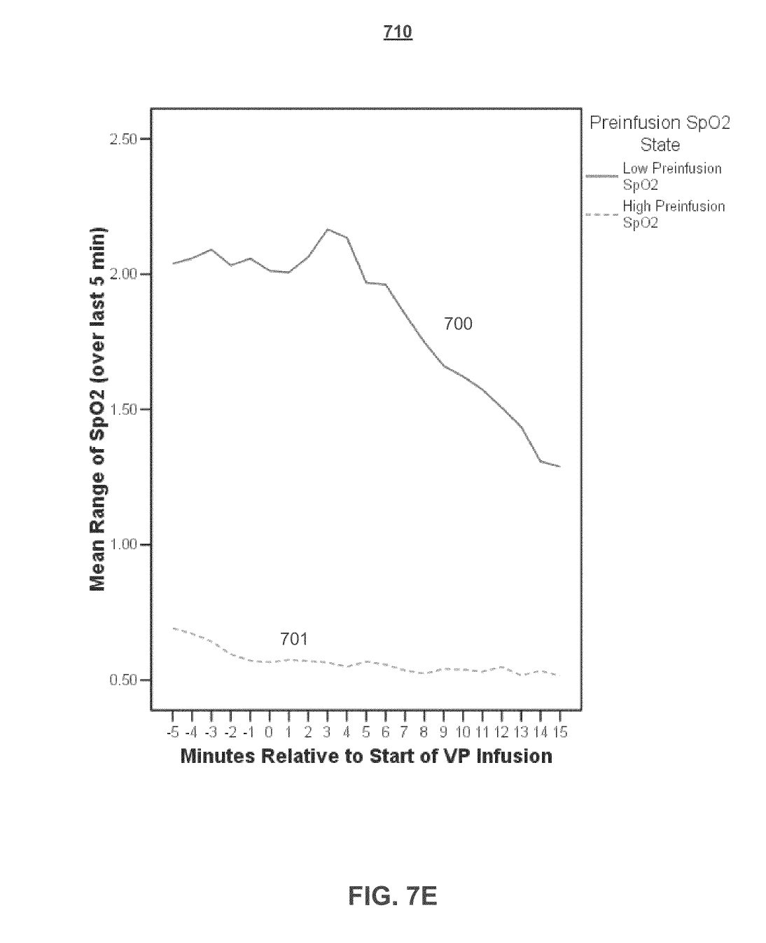

FIGS. 7A-7E illustrate an example for use of system 400, described above, to identify potential hypoperfusion, as a means to alert clinicians to a potentially untoward or fatal condition. Adequate oxygen delivery and clearance of cellular waste are needed to maintain healthy cellular function. In normal conditions with adequate respiration and blood volume, tissue perfusion is adequate to deliver oxygen and other nutrients to cells and to remove cellular waste. If blood pressure or circulating blood volume is inadequate, then perfusion of tissues will be inadequate, with demand exceeding supply and with toxic wastes remaining in tissues. Hypoperfusion leads to cellular dysfunction, accumulation and release of cytotoxic substances, and eventual cellular death when the condition persists. On the patient level, sustained systemic hypoperfusion during surgery is a risk factor for untoward events, including diminished organ function, delayed wound healing, and increased likelihood of infection. Early detection of periods of hypoperfusion may enable earlier treatment and improve patient outcomes.

A relationship which occurs in patients undergoing surgery with anesthesia exists between postoperative mortality and a “Triple Low” condition of Low BIS, Low MAP and Low MAC (i.e. Bispectral Index™ (BIS<45), mean arterial pressure (MAP)<75 mmHg, and end-tidal volatile anesthetic concentrations in MAC-equivalents (MAC)<0.70.) Patients who are in this Triple Low state have BIS values which are less than that which would be expected based only upon the anesthetic concentration used. Because BIS values correlate with levels of cerebral metabolism, lower than expected BIS values may be due to other conditions that decrease metabolism (e.g., cooling, dementia, hypoperfusion, hypoglycemia etc.). Because patients in the Triple Low condition demonstrate an increase in BIS values in response to increasing blood pressure following vasopressor administration, the Triple Low condition may be a marker of hypoperfusion. Vasopressor treatment of hypotension may improve outcomes. For example, patients who received vasopressor treatment within 5 minutes of entering a Triple Low state had a lower 90-day mortality rate compared to those who received vasopressors later or not at all (2.0% vs. 2.9%). Thus, early detection of hypoperfusion may allow earlier interventions that improve patient outcomes.

System 400 (FIG. 4) may be used to identify potential instances of hypoperfusion as a means to alert clinicians to this potentially untoward condition using physiological parameters such as SpO2, variability of SpO2, brain state, blood pressure and other suitable physiological parameters, or a combination of physiological parameters. FIGS. 7A-7E show, among other things, that patients who have probable hypoperfusion demonstrate an increase in BIS and SpO2 following onset of vasopressor infusion.

FIGS. 7A-7E show illustrative plots of group-average parameter values that were plotted as a function of time relative to the start of vasopressor infusion (i.e., the infusion began at time 0) where the patients are grouped based on the patient's SpO2 level within one minute immediately prior to starting the infusion (i.e., higher or lower than the 25th percentile of the population SpO2 value of 97%). The data represents patient information including responses to sustained vasopressor treatment. The subset of patients who received infusions of phenylephrine for 15 minutes or longer included approximately n=1684 patients.

In each plot of FIGS. 7A-7E, curve 700 was generated from approximately the 25% of the patients who had lower SpO2 levels prior to infusion, and curve 701 was generated from approximately the 75% of the patients who had higher SpO2 levels prior to infusion. It should be noted that the patients grouped as “Low SpO2” preinfusion did not exhibit SpO2 levels that are considered abnormally low, but rather, would likely be considered acceptable in current clinical practice. As will be illustrated with respect to the discussion of FIGS. 7A-7E, the patients grouped as “Low SpO2” may also be considered to have exhibited a “masked low” SpO2 condition. These masked low patients had SpO2 levels that are considered acceptable in current clinical practice. However, these patients exhibited indications of hypoperfusion. Even though these masked low patients were found mostly in the low SpO2 group, it should be understood that a patient may have a masked low SpO2 level irrespective of the patient's measured SpO2 value. For example, under normal circumstances, acceptable SpO2 values may range from 94-99%. In FIGS. 7A-7E, the High SpO2 group had SpO2 values greater than 97% and the Low SpO2 group had SpO2 values between 95% and 97%.

FIG. 7A illustrates that the agent concentration was roughly constant over the observation period (from 5 minutes before starting the infusion to 15 minutes after infusion onset). Anesthetic agents are typically titrated so patients are maintained intraoperatively at the same physiological state. Patients in the Low SpO2 group received less anesthetic agent (i.e., they were maintained at lower mean MAC Equivalent concentrations), implying that they were more sensitive to the agent than those in the High SpO2 group.

FIG. 7B demonstrates that both groups of patients had a drop in mean arterial pressure prior to starting the vasopressor infusion; however, the pressure went lower in the Low SpO2 group. Both groups of patients generated a good response (increased MAP) to vasopressor treatment.

As shown in FIG. 7C, although the agent concentration is relatively constant (FIG. 7A) and the blood pressure response to treatment was similar between groups (FIG. 7B), the Low SpO2 patients had a significantly greater increase in BIS (4 BIS points over 20 minutes) in response to vasopressor treatment. This may indicate that the brains of the masked low SpO2 patients in this group were previously hypoperfused and were exhibiting a relative reduction in metabolism (i.e., a lower-than-expected BIS for the anesthetic state). These masked low patients returned (increased) to the BIS range normally associated with the anesthetic effect of the current MAC-level of anesthetic agent after resolving the additional depression in BIS secondary to hypoperfusion.

As illustrated in FIG. 7D, the SpO2 level in the masked low patients in the Low SpO2 group dropped in concert with decreasing blood pressure prior to vasopressor treatment, and then increased in response to treatment. There is a lack of change in SpO2 value before or after vasopressor treatment in patients grouped as High SpO2. The change in systemic arterial saturation with change in blood pressure may indicate that the masked low patients were hypoperfused prior to vasopressor intervention. Because the patients in the masked low condition had clinically acceptable SpO2 levels, their hypoperfused conditions were masked by their clinically normal SpO2 levels.

FIG. 7E shows that the mean Range of SpO2 (calculated as Maximum SpO2−Minimum SpO2 over a moving 5 minute window) is much greater in patients who have Low SpO2 prior to treatment. The SpO2 Range decreases following vasopressor administration because the SpO2 is increasing (and becoming stable) in masked low patients. (Although SpO2 and SpO2 Range are negatively correlated (R=−0.458, p<0.001, N=23,999), they provide complementary information. Hence, each of the SpO2 and SpO2 Range, and their combination, may be independent predictors of 90-day mortality, as demonstrated in FIG. 8 and the related description below using Cox Proportional Hazards Modeling.

The occurrence of a “Triple Low” (i.e., Low BIS, Low MAP, Low MAC) may be used as a potential marker to identify of hypoperfusion. Additionally, SpO2 parameters (i.e., case-average SpO2, case-average SpO2 Range, and time within a case that SpO2 Range exceeds a threshold of 2% (e.g., HoursSpO2RangeGT2)) may be used to identify hypoperfusion in the masked low patients, which is a risk factor for worse postoperative morbidity and mortality.

For example, Cox models may be used to demonstrate that each of case-average SpO2, case-average SpO2 Range, and HoursSpO2RangeGT2 are independent predictors of 90-day mortality, after controlling for case-average BIS, MAP, MAC and baseline demographic (age, sex, race, body mass index) characteristics and morbidity and procedural risk. Lower SpO2, higher SpO2 Range, and longer HoursSpO2RangeGT2 may represent an increased risk of 90-day postoperative mortality. Consequently, SpO2 parameters may provide additional information about the patient's risk profile alone or in addition to the parameters used to identify a Triple Low state (of Low BIS, Low MAP, and Low MAC) and patient demographic characteristics. Indeed, and by way of example, lower SpO2, higher SpO2 Range, and greater HoursSPO2RangeGT2 may increase the risk of 90-day postoperative mortality by 10% per percent saturation less than 100, 16% per percent range of saturation greater than 0, and 74% per hour spent with SpO2>2% respectively (hazard ratios of 0.90 (p<0.001), 1.16 (p=0.002), 1.74 (p<0.001)).

As shown in FIG. 8, patients whose average clinical state had low MAP and high variability in SpO2 (e.g., Hi SpO2 Range) were significantly more likely to die postoperatively compared to the reference group who had average BIS, MAP and SpO2 Range values near the population mean for each. The risk of mortality was greater still for patients who also had low case-average BIS values.

FIG. 8 shows an illustrative example display of the relative risk hazards for each patient state based on various physiological parameters according to an embodiment. In the illustrative examples described in FIG. 8, the monitoring system monitors and provides risk assessment information based on three physiological parameters. The three physiological parameters include a measure of brain state (e.g., consciousness and sedation) such as the Bispectral Index™ (BIS™), a measure of blood pressure such as mean arterial pressure (MAP), and a measure of oxygen saturation, such as SpO2 or SpO2 variance. It will be understood by those of skill in the art that any other suitable patient information may be used to provide risk assessment information (e.g., heart rate (HR), respiratory rate, blood pressure, Systolic Pressure, Diastolic Pressure, as well as other derived hemodynamic parameters (ratios, products or differences of heart rate and the components of BP e.g., Systolic/Diastolic or MAP/HR), temperature, ScO2, rSO2, etc.). Furthermore, while the illustrative patient risk assessment display described in FIG. 8 shows patient state and risk assessment information based on these three physiological parameters, it will be understood that any number of patient information variables may be used by the monitoring system to generate risk-assessment information and alerts.

The following example will illustrate the operation of patient monitoring system 400 in accordance with an embodiment. A data set of patient physiological characteristics may be obtained from one or more sensors capable of collecting physiological data from a patient. The patient physiological data includes, for example, intra-operative data such as minute-by-minute measurements of: brain state, blood pressure (systolic, diastolic, MAP), oxygen saturation, heart rate, the anesthetic agent concentrations being used (delivered or expired), and other drugs that were given (e.g., muscle relaxants, analgesics, etc.).

The physiological data may be used to calculate one or more physiological parameters, as described with respect to flow chart 500 (FIG. 5). The present embodiment monitors physiological parameters MAP, SpO2 Range, and BIS as measures of patient physiological data. Other embodiments may derive patient states and risk assessment information using other physiological parameters, including: other measures of hemodynamic state and cardiovascular function (e.g., heart rate, diastolic pressure, systolic pressure, SpO2 variance, stroke volume, cardiac output and flow), other brain monitoring measurements, as well as other measures of patient brain state.

In the present embodiment, physiological parameters may be calculated at step 504 (FIG. 5) using patient monitoring system 400 (FIG. 4). In some embodiments, physiological data may be pre-determined or pre-recorded and may be input to patient monitoring system 400 (FIG. 4).

In addition to the reference state, eight additional patient states may be defined by being outside of the reference state and being either higher or lower than the population mean of MAP, SpO2 and BIS. As illustrated in Table 1 above, patient states may be defined based on the sections of the population that do not fall within a reference group, as either being high or low relative to the reference population, thus creating eight cells. These eight cells may also be represented as part of a three-dimensional cube (FIG. 6, display 602) or two-dimensional squares (FIG. 6, 604). The patient state may be defined by where the patient falls, either higher or lower than a reference population for each of the evaluated parameters. In addition to these eight patient states, a ninth patient state may be defined in which the patient falls within a reference population for all of the evaluated parameters. The reference state may be the condition when the patient is within 0.75 standard deviations of the mean of each parameter (e.g., BIS, MAP and SpO2). In certain embodiments, if any of the BIS, MAP and SpO2 are outside 0.75 SD away from their respective mean, then they are in one of the 8 other states (listed in Table 1 above).

Each patient state may have one or more associated hazard ratios derived from a model. FIG. 8 includes illustrative two-dimensional patient risk assessment charts including associated hazard ratio parameters. These hazard ratios may be calculated using a proportional hazards model such as, for example, the Cox proportional hazards model. The Cox proportional hazards model may be used to calculate the relative risk of a given endpoint relative to the patients treated in the reference population. The presence of an asterisk after the hazard ratios for each of the patient states indicates which of the calculated risks or hazard ratios are statistically different from that of the reference population (p<0.005). A hazard ratio greater than 1 indicates an increased likelihood of the given event (i.e., endpoint) happening. A hazard ratio less than 1 indicates a decreased likelihood of the given event (i.e., endpoint) happening.

After patient physiological data is collected and the patient is classified into one or more patient states (e.g., at steps 502 and 504 of FIG. 5), the patient state information may be displayed on display 402 of system 400. Patient state information for each of the monitored physiological parameters may be defined based on the distribution of the reference population for each state. In certain embodiments, the current patient state for a patient may be visually distinguished (e.g., highlighted) to indicate the patient state to a physician.

After patient state information is determined (and displayed), the risk associated with the chosen endpoint(s) may be calculated (and displayed). In the example illustrated in FIG. 8, the endpoint chosen is patient mortality rate. Patient state information may be analyzed to determine the relative risk of death for a patient, relative to the reference population, within various time periods after a particular procedure: in-hospital, 30-days, 90-days and 1-yr. The reference population incidence of mortality, in this example, for in-hospital is 0.5%, for 30 days: 0.8%, for 90 days: 1.8% and for 1 year: 4.8%. A Cox proportional hazards model may be used to derive the relative risk of mortality at each of the mortality time points using the average BIS, average MAP, and SpO2 Range measures. The relative risk (hazard ratio) of each mortality endpoint is calculated for each patient state and displayed within associated patient state cells in FIG. 8.

After the hazard ratios are calculated, the ratios may be analyzed to determine if the relative risk of mortality at each of the patient states is materially different from the reference population (p<0.05). In the example illustrated in FIG. 8, patient states 802, 804, 806 and 808 are examples of undesirable patient states in terms of mortality, as indicated by the relatively high hazard ratio of 2.46, 2.18, 2.22 and 1.49, respectively. The patients whose measured BIS, MAP and SpO2 Range values place them in these states may have a higher risk of mortality than those patients whose measured BIS, MAP and SpO2 Range values place them in the reference population. This information may be used in a patient monitor (e.g., patient monitoring system 400) to calculate patient state information and to display risk assessment information. For example, the patient monitor may be configured to alert a physician if the patient transitions into or is in one of the undesirable (high-risk) states for more than a predetermined period of time. In the example of FIG. 8, the least desirable states for each time interval (30-day, 90-day, etc.) are, for example, patient states 802, 804, 806 and 808. The physician may then intervene to adjust the patient's parameters and drive the patient into a more desirable state. In some embodiments, the patient monitor may only display the current patient state information and an indication of the relative risk associated with that patient state. The patient monitor may also display information about the relative risks of other patient states and/or information about changes in one or more physiological parameters that may lead to a change in the patient state.

FIGS. 7A-7E demonstrate that patients whose average SpO2 was in the lower quartile immediately prior to vasopressor infusion (<97%) exhibited SpO2 and BIS responses to vasopressor treatment, indicating that these patients were likely hypoperfused. In addition, the analyses above (FIG. 8) demonstrate that: patients with higher than average SpO2 Range and lower than average MAP had worse postoperative mortality, especially those patients with lower than average BIS values.

System 400 may use parameters of SpO2 variability (with or without further clinical parameters) as well as other estimates of systemic and cerebral perfusion for the real-time detection of untoward states including hypoperfusion, inadequate metabolism, or elimination of cellular toxins. These SpO2 variability parameters may be used to determine whether patients are hypoperfused. Upon detection of these states, system 400 may provide an alert or alarm to notify clinicians of the potential need to intervene. These SpO2 variability parameters may represent relatively small changes in SpO2 values (e.g., 2%) relative to a normal baseline SpO2 value (e.g., an SpO2 value between 94-99%).

When patients are conscious, BIS monitoring is typically not used since it is not necessary to monitor their sedative/hypnotic state. In an embodiment, the system may be adapted for use in a patient care setting in which BIS monitoring is not available or not in use, such as a hospital general care floor, an emergency room. A monitoring system in which the inputs are SpO2 and a hemodynamic parameter may be used to monitor patients for the occurrence of a risk state based on two parameters (e.g., low MAP and high SpO2 Range). In this embodiment, the parameter derived from SpO2 may be one of SpO2, SpO2 Range (calculated over the recent history (e.g., 15 min)), and Time SpO2 Range>2 (calculated over the recent history (e.g., 15 min)). In this embodiment, the hemodynamic parameter may be one of systolic blood pressure, diastolic blood pressure, MAP, HR or MAP/HR.

While the disclosure may be susceptible to various modifications and alternative forms, specific embodiments have been shown by way of example in the drawings and have been described in detail herein. However, it should be understood that the foregoing is merely illustrative of the principles of this disclosure and various modifications can be made by those skilled in the art without departing from the scope and spirit of the disclosure.

What is claimed is:

1. A medical monitoring system comprising:
an input configured to receive a plethysmographic signal from a subject;
a processor configured to calculate an oxygen saturation value based on the plethysmographic signal and classify the subject's state based on a high or low oxygen saturation value in combination with a high or low value of a different physiological parameter; and
a display configured to show the classified state of the subject and an amount of time the subject has been in the classified state.

2. The medical monitoring system of claim 1, wherein the different physiological parameter comprises a depth of anesthesia value.

3. The medical monitoring system of claim 1, wherein the different physiological parameter comprises a mean arterial blood pressure value.

4. The medical monitoring system of claim 1, further comprising an alert triggered when the amount of time passes a threshold.

5. The medical monitoring system of claim 1, wherein the display comprises a grid graphically representing a plurality of subject states, and identifying the classified state of the subject.

6. The medical monitoring system of claim 1, wherein the processor is configured to classify the subject's state based on the high or low oxygen saturation value in combination with the high or low value of the first different physiological parameter and further in combination with a high or low value of a second different physiological parameter.

7. The medical monitoring system of claim 1, wherein the display is further configured to show a risk that the subject will reach an identified endpoint, per minute of time the subject remains in the classified state.

8. The medical monitoring system of claim 1, wherein the classified state comprises a double low state comprising a low oxygen saturation value and a low level of consciousness value.

9. The medical monitoring system of claim 1, wherein the classified state comprises a triple low state comprising a low oxygen saturation value, a low level of consciousness value, and a low mean arterial pressure value.

10. The medical monitoring system of claim 1, wherein the processor is further configured to trigger an alert based on the classified state.

11. The medical monitoring system of claim 1, wherein the processor is further configured to compare the oxygen saturation value to a population average oxygen saturation value.

12. The medical monitoring system of claim 1, wherein the processor is further configured to calculate a reference set based on the plethysmographic signal, wherein the reference set defines at least one reference state.

13. The medical monitoring system of claim 12, wherein the processor is further configured to compare the oxygen saturation value to the at least one reference state and classify the subject's state based at least in part on the comparison.

14. The medical monitoring system of claim 1, wherein the display is further configured to show the oxygen saturation value.

15. The medical monitoring system of claim 1, wherein the processor is configured to provide an indication of at least one change that may be made, wherein the change may cause the subject to enter a more desirable state.

16. A method of prompting medical intervention using classified subject states, comprising:
receiving a plethysmographic signal from a subject;
calculating, using a processor, an oxygen saturation value based on the plethysmographic signal;
classifying, using a processor, the subject's state based on a high or low oxygen saturation value in combination with a high or low value of a different physiological parameter
displaying, using a display, the classified state of the subject and an amount of time the subject has been in the classified state.

17. The method of claim 16, further comprising prompting a user to intervene to change the subject's classified state.

18. The method of claim 16, wherein the different physiological parameter comprises a depth of anesthesia value.

19. The method of claim 16, wherein the different physiological parameter comprises a mean arterial blood pressure value.

20. The method of claim 16, further comprising triggering an alert when the amount of time passes a threshold.
